(19) United States
(12) Patent Application Publication
(10) Pub. No.: US 2004/0133070 A1
(43) Pub. Date: Jul. 8, 2004

(54) Condom device

(76) Inventor: Jerome Lin, Yung Ho City (TW)

Correspondence Address: BACON & THOMAS, PLLC, 625 SLATERS LANE, FOURTH FLOOR, ALEXANDRIA, VA, 22314

(21) Appl. No.: 10/337,770

(22) Filed: Jan. 8, 2003

(51) Int. Cl. A61F 5/00

(52) U.S. Cl. 600/38

(57) ABSTRACT

The invention discloses a condom device including a broadened sheath from a common condom for containing a miniature motor, batteries and conductive pieces. The condom covers and wraps the sheath within after forming an integral from extrusion. The batteries form conductive loops by connecting the conductive pieces. The motor is in contact with the conductive pieces such that the condom produces vibration when the batteries provide power supply for activating the motor. Therefore pleasure is achieved during sexual activities for further increasing the willingness of condom usage, thereby preventing venereal diseases and AIDS resulted from inappropriate sexual activities.

### BACKGROUND OF THE INVENTION

(a) Field of the Invention

The invention relates to a condom device, and more particularly, to a condom device provided with an broadened sheath having a miniature motor and batteries for increasing the pleasure and encouraging the use of condoms during sexual activities, thereby effectively preventing the spreading of venereal diseases and AIDS.

(b) Description of the Related Art

According to statistics, the youngster and the middle age account for the majority of AIDS patients worldwide. Among those, young people between the ages of 15 to 20-something make up one-fifth of the total volume. And in Taiwan, up to the end of September this year, the number of AIDS patients has reached as high as 3,422, wherein 90 percent have been infected through sexual activities, and nearly 40 percent are youngsters between the ages of 20 to 29. The results clearly point out that young people yet lack correct and mature sexual acknowledgement regardless of the openness of sexual perception and widespread sexual knowledge; only temporary happiness and satisfaction are being pursued instead. Although Disease Control Bureau of Health Department in Taiwan has persistently promoted AIDS prevention in connection with those young people in the hope that they use condoms during sexual activities, the results have turned out to be unsatisfactory.

### SUMMARY OF THE INVENTION

Therefore, the object of the invention is to provide a condom device, wherein a sheath of a prior condom is broadened for accommodating a miniature motor, batteries and conducting pieces. When the condom device is not in use, power supply between the motor and the conducting pieces is cut off using an insulating piece made of a cotton thread. When the condom device is to be used, the insulating piece is removed to connect to the power supply of the motor for providing the condom with vibration, thereby increasing the pleasure of sexual activities.

The other object of the invention is to provide a condom device having a protruding edge at the outer periphery of the sheath thereof, and the protruding edge is for rubbing against the clitoris (the G-spot) of a female to further stimulate erogenous zones of the female.

### BRIEF DESCRIPTION OF THE DRAWINGS

FIG. 1 is a conventional schematic view showing the appearance according to the invention.

FIG. 2 is a cross sectional and elevational view according to the invention.

FIG. 3 is an enlarged partial view showing the insulating piece according to the invention.

FIG. 4 is another sectional and elevational view according to the invention.

FIG. 5 is a conventional schematic view showing the appearance of a rolled up condom device according to the invention.

### DESCRIPTION OF THE PREFERRED EMBODIMENTS

Detailed descriptions of the preferred embodiments according to the invention shall be given with the accompanying drawings hereunder.

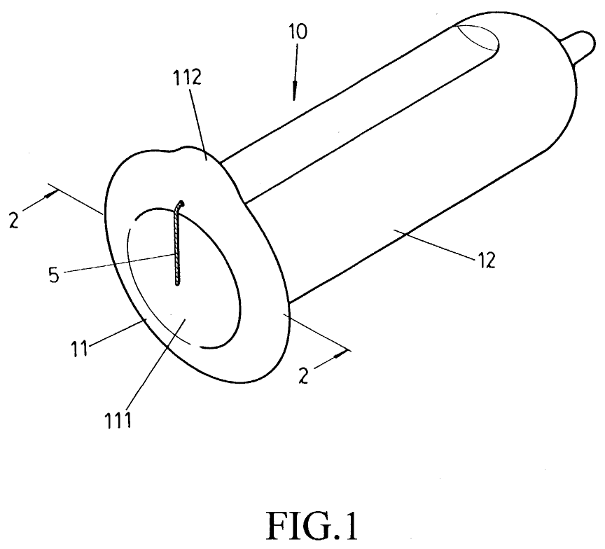

Referring to FIGS. 1 to 5 in accordance with the invention, a condom 10 comprises a sheath 11 and a pouch portion 12, wherein an opening 111 of the sheath 11 is for accommodating the penis of a male into the pouch portion 12.

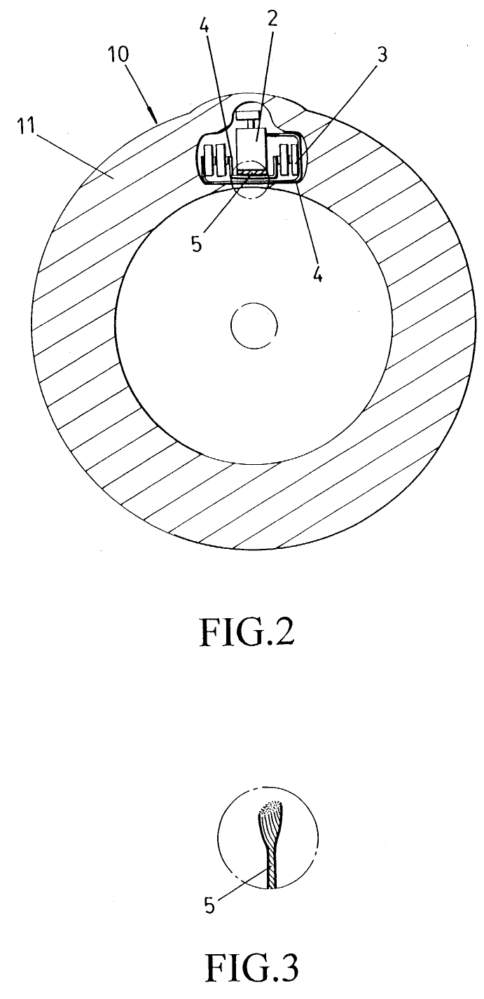

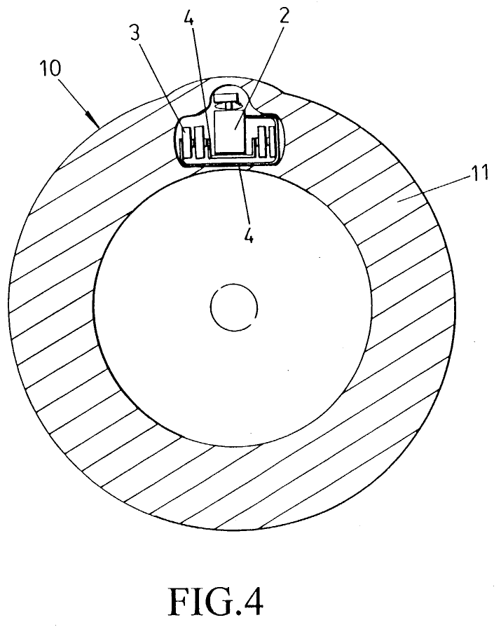

The characteristics of the above structure are that the sheath 11 is broadened for containing a miniature motor 2, batteries 3 and conducting pieces 4. After forming an integral from extrusion, the condom 10 covers and wraps the sheath 11 within to form one body. Wherein, the motor 2 is covered at one side of the sheath 11; on the two sides of the motor are disposed with two batteries 3, respectively; the batteries 3 are connected in series by means of a conducting piece 4 to form a conductive loop, respectively; the batteries 3 are consisted of a plurality of miniature mercury cells connected in series; the motor 2 is in contact with the conductive piece 4 above, and between the two is a space for inserting an insulating piece 5 made from a flattened cotton thread (as shown in FIG. 2). When the condom is not in use, the insulating piece 5 cuts off the power supply of the motor 2; when the condom is to be used, the user may pull out the insulating piece 5 to electrically connect the motor 2 and the conducting pieces 4 (as shown in FIG. 4) such that the miniature motor 2 is activated for vibration that increase the pleasure of sexual activities.

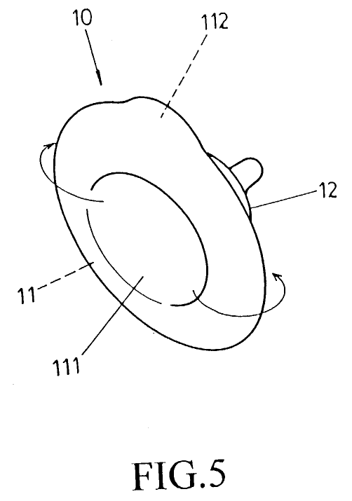

In addition, a protruding edge 112 is provided at the outer periphery of the sheath 11, and the protruding edge is for rubbing against the clitoris (the G-spot) of a female during sexual activities to further stimulate erogenous zones of the female. Also, referring to FIG. 5, the condom 10 in accordance with the invention may be rolled up for easier storage.

Conclusive from the above, the invention capable of increasing the frequency of condom usage while making sexual activities a pleasure for both sexes, thereby preventing venereal diseases and AIDS resulted from inappropriate sexual activities.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. A condom device comprising a condom consisted of a sheath and a pouch portion; and the characteristics thereof are that the sheath is broadened for containing a motor, a plurality of batteries and conducting pieces; the batteries form conductive loops by connecting the conductive pieces; the motor is in contact with the conductive pieces such that the condom produces vibration when the batteries provide power supply for activating the motor.

2. The condom device in accordance with claim 1, wherein the motor and the conductive pieces are insulated by means of an insulating piece when the condom is not in use.

3. The condom device in accordance with claim 1, wherein a protruding edge is provided at the outer periphery of the sheath for stimulating the clitoris of a female.
